(19) United States
(12) Patent Application Publication
(10) Pub. No.: US 2025/0195846 A1
(43) Pub. Date: Jun. 19, 2025

(54) GUIDE WIRE INSERTION METHOD

(71) Applicant: ASAHI INTECC CO., LTD., Seto-shi (JP)

(72) Inventors: Keisuke Ushida, Seto-shi (JP); Narumi Hoshino, Seto-shi (JP); Sora Chikaoka, Seto-shi (JP)

(73) Assignee: ASAHI INTECC CO., LTD., Seto-shi (JP)

(21) Appl. No.: 18/752,084

(22) Filed: Jun. 24, 2024

(30) Foreign Application Priority Data
Dec. 13, 2023 (JP) 2023-210608

(51) Int. Cl. A61M 25/09

(52) U.S. Cl. CPC A61M 25/09; A61M 2025/09083; A61M 2025/09133

(57) ABSTRACT

A guide wire insertion method for inserting a guide wire into a blood vessel having a main vessel and a side branch branched from the main vessel. The method includes providing a guide wire having a knuckle advance suppressing structure; inserting the guide wire into a main vessel of a blood vessel of a patient and pushing a distal end of the guide wire toward an affected part; after the distal end of the guide wire is caught by a side branch of the blood vessel, generating a knuckle at a distal end portion of the guide wire, and removing the distal end of the guide wire from the side branch, and pushing the distal end portion toward the affected part in the main vessel while maintaining the knuckle.

### CROSS-REFERENCE TO RELATED APPLICATION

This application claims priority to Japanese Patent Application No. 2023-210608, filed on Dec. 13, 2023, the contents of which are incorporated by reference herein in their entirety.

### TECHNICAL FIELD

The present disclosure relates to a method for inserting a guide wire into a blood vessel during vascular treatment.

### BACKGROUND ART

A guide wire is known as a medical device for guiding a catheter-like medical tool, which is inserted into a tubular organ of a human body, such as a blood vessel or a digestive organ, to a target site.

Percutaneous coronary intervention (PCI) is performed for treatment of ischemic cardiac disease in a cardiac coronary artery, such as cardiac angina and myocardial infarction. In the percutaneous coronary intervention, a guide wire is inserted into a blood vessel to pass through a disease-causing constricted part (affected part) and a balloon catheter or a stent is delivered to the constricted part using the guide wire.

Conventionally, in the PCI technique, a branching blood vessel branched from a main vessel of a blood vessel is selected, and a guide wire is inserted into the branching blood vessel. For this reason, generally, a guide wire having a shaped distal end is used and the distal end is operated and guided to an affected part. For example, Japanese Unexamined Patent Publication No. S 59-067968 discloses vascular treatment using a guide wire having a shaped distal end.

### SUMMARY

However, as in main vessel treatment, there are many cases in which it is not necessary to select a branching blood vessel. In such main vessel treatment, when a distal end of a guide wire is operated to be pushed forward in a blood vessel, there are problems in which the distal end of the guide wire unintentionally migrates into a microscopic blood vessel or the distal end comes into contact with a blood vessel wall to cause dissociation or perforation. In addition to such problems, there is also a concern about an increase in the load applied to the blood vessel wall due to the point contact of the distal end of the guide wire with the blood vessel wall.

The present disclosure has been made in view of the above-described circumstances, and an object of the present disclosure is to provide a guide wire insertion method by which a distal end of a guide wire is less likely to migrate into a microscopic blood vessel or cause dissociation or perforation of a blood vessel wall. Another object is to suppress the load applied to the blood vessel wall.

In one aspect, the present disclosure provides a guide wire insertion method for inserting a guide wire into a blood vessel having a main vessel and a side branch branched from the main vessel, the method including: providing a guide wire having a knuckle advance suppressing structure; inserting the guide wire into a main vessel of a blood vessel of a patient and pushing a distal end of the guide wire toward an affected part; after the distal end of the guide wire is caught by a side branch of the blood vessel, generating a knuckle at a distal end portion of the guide wire; and removing the distal end of the guide wire from the side branch, and pushing the distal end portion toward the affected part in the main vessel while maintaining the knuckle. The knuckle advance suppressing structure is configured to suppress further advancement of the knuckle as the distal end portion is pushed toward the affected part in the main vessel. (Disclosure 1).

### BRIEF DESCRIPTION OF DRAWINGS

FIG. 1 is a cross-sectional view illustrating a structure of a guide wire used in a guide wire insertion method according to an embodiment of the present disclosure.

FIG. 2 is a cross-sectional view illustrating a modification of the structure of the guide wire used in the guide wire insertion method.

FIG. 3 is a flowchart illustrating a flow of a guide wire insertion method.

FIG. 4 is a schematic diagram (No. 1) illustrating a process of a guide wire insertion method.

FIG. 5 is a schematic diagram (No. 2) illustrating the process of the guide wire insertion method.

FIG. 6 is a schematic diagram (No. 3) illustrating the process of the guide wire insertion method.

### DESCRIPTION OF EMBODIMENTS

Hereinafter, a guide wire insertion method according to an embodiment of the present disclosure will be described. The present disclosure is not limited to the embodiments described below, and the described embodiments are merely examples for describing the technical features of the present disclosure. The shapes and dimensions illustrated in the drawings are merely illustrated to facilitate understanding of the contents of the present disclosure, and do not accurately reflect actual shapes and dimensions.

### <Structure of Guide Wire>

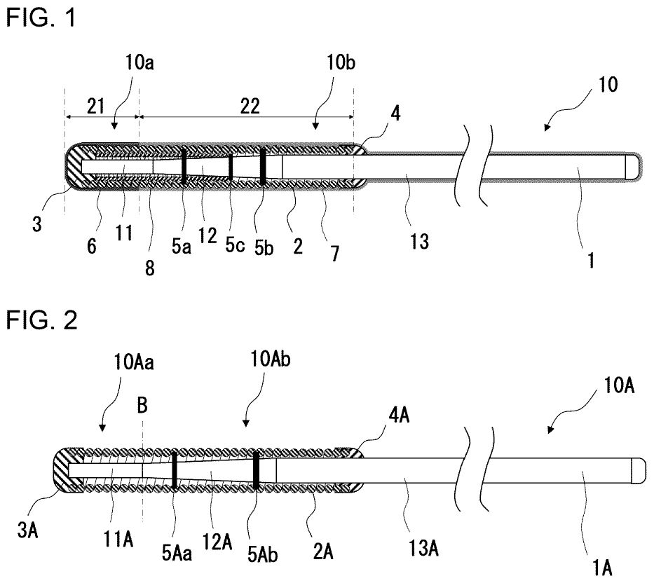

Since a guide wire insertion method of the present disclosure uses a guide wire having a knuckle advance suppressing structure, first, a structure of a guide wire 10 used in the guide wire insertion method will be described with reference to FIGS. 1 and 2. The guide wire 10 is suitable for treatment of ischemic cardiac disease in a cardiac coronary artery, and is a workhorse guide wire that guides a catheter, a stent, or the like for percutaneous coronary intervention (PCI) to a constricted part in a coronary artery. A distal end side of the guide wire 10 is a side to be inserted into a body, and a proximal end side of the guide wire 10 is a side to be operated by a professional such as a doctor. In the guide wire 10, a surface of a first area 10a located on the distal end side of the guide wire 10 is less slippery than a surface of a second area 10b located closer to the proximal end side than the first area, so that the knuckle advance suppressing structure (details are described below) is realized.

In the present description, the “distal end side” means a side that is located in a direction along an axial direction of the guide wire and a direction in which the guide wire proceeds toward a target site. The “proximal end side” means a side that is located in a direction along the axial direction of the guide wire and in a direction opposite to the above-described distal end side. The term “distal end” refers to an end on the distal end side in any given member or part, and the term “proximal end” refers to an end on the proximal end side in any given member or part. A “distal end portion” refers to, in any given member or part, a portion including the distal end and extending from the distal end toward the proximal end side to the middle of the member or the like. A “proximal end portion” refers to, in any given member or part, a portion including the proximal end and extending from the proximal end toward the distal end side to the middle of the member or the like. In FIG. 1, the left side in the drawing is the “distal end side” to be inserted into a body, and the right side in the drawing is the “proximal end side” to be operated by a professional.

When a guide wire is inserted into a blood vessel or the like, in order to improve the passability through a lesion in the blood vessel or the like, or in order to prevent damage of a blood vessel wall or the like and unintentional migration into a side branch, the distal end side of the guide wire may be intentionally bent into a U-shape in the blood vessel. This state is referred to as a knuckle (or prolapse). After the knuckle is formed, when the knuckle further advances (i.e., the length of the knuckle increases) as the guide wire is pushed forward, the reaction force of the guide wire trying to return to a straight state increases, which may cause a defect such as the damage of the blood vessel wall or the like. A structure for suppressing excessive advance of the knuckle by changing the rigidity or the surface property on the distal end side of the guide wire is referred to as the knuckle advance suppressing structure.

FIG. 1 is an explanatory view illustrating the structure of the guide wire 10. The guide wire 10 includes an elongated core shaft 1 and a tubular body 2 provided on the outer side of the core shaft 1. A distal tip 3 for joining the core shaft 1 and the tubular body 2 is provided at the distal end of the guide wire 10. A fixation portion 4 for fixing the core shaft 1 and the tubular body 2 is provided at the proximal end of the tubular body 2. An inner tubular body 8 is arranged along the outer periphery of the core shaft 1 on the inner side of the tubular body 2.

The core shaft 1 is an elongated member serving as a shaft of the guide wire 10. As illustrated in FIG. 1, the core shaft 1 has a small diameter portion 11 on the distal end side and a large diameter portion 13 on the proximal end side. The core shaft 1 has a tapered portion 12 between the small diameter portion 11 and the large diameter portion 13. The outer diameter of the tapered portion 12 decreases from the proximal end side toward the distal end side. The core shaft 1 can be formed of, for example, materials such as stainless alloys (SUS302, SUS304, SUS316, and the like), superelastic alloys such as a Ni—Ti alloy, a piano wire, a nickel-chromium alloy, a cobalt alloy, and tungsten. The material of the core shaft 1 is not limited thereto, and the core shaft 1 may be formed of other known materials as long as the core shaft 1 itself can be prevented from being cut and the distal end portion can be rotated.

The small diameter portion 11 has a columnar shape in which the outer diameter is constant from the distal end to the proximal end. Alternatively, the small diameter portion 11 may have a flat cross-sectional shape by press working. The large diameter portion 13 has a columnar shape in which the outer diameter is constant from the distal end to the proximal end. The tapered portion 12 has a circular truncated cone shape in which the outer diameter is gradually enlarged from the distal end toward the proximal end.

The tubular body 2 is wound around the core shaft 1 so as to cover the outer periphery of the small diameter portion 11, the tapered portion 12, and a part of the large diameter portion 13 of the core shaft 1.

The tubular body 2 may be a single coil formed into a hollow cylindrical shape by spirally winding one wire having a circular cross-section, or may be a hollow twisted wire coil formed into a hollow cylindrical shape by a twisted wire that is obtained by twisting a plurality of wires. The tubular body 2 may be configured by combining a single coil and a hollow twisted wire coil. The tubular body 2 can be formed of, for example, stainless alloys (SUS302, SUS304, SUS316, and the like), superelastic alloys such as a Ni—Ti alloy, a piano wire, a nickel-chromium alloy, radiotransparent alloys such as a cobalt alloy, gold, platinum, tungsten, and radiopaque alloys such as an alloy containing these elements (for example, a platinum-nickel alloy). The material of the tubular body 2 is not limited thereto, and may be formed of known materials other than the above-described materials. In the present embodiment, the tubular body 2 is formed of a radiopaque alloy on the distal end side and a stainless alloy on the proximal end side, and the outer diameter thereof is configured to be substantially constant from the distal end to the proximal end.

The distal tip 3 for joining the core shaft 1 and the tubular body 2 is formed at the distal end of the guide wire 10, that is, the distal end of the core shaft 1. The distal tip 3 is formed of a metal solder such as a silver solder, a gold solder, zinc, a Sn—Ag alloy, or an Au—Sn alloy, and the distal end of the core shaft 1 and the distal end of the tubular body 2 are fixed to each other by the metal solder. The distal tip 3 may be formed of an adhesive such as an epoxy adhesive, and the distal end of the core shaft 1 and the distal end of the tubular body 2 may be fixed to each other by the adhesive.

The fixation portion 4 for fixing the core shaft 1 and the tubular body 2 is formed at the proximal end of the tubular body 2. The fixation portion 4 is formed of a metal solder such as a silver solder, a gold solder, zinc, a Sn—Ag alloy, or an Au—Sn alloy, and the proximal end of the tubular body 2 is fixed to the large diameter portion 13 of the core shaft 1 by the metal solder. The fixation portion 4 may be formed of an adhesive such as an epoxy adhesive, and the large diameter portion 13 of the core shaft 1 and the proximal end of the tubular body 2 may be fixed to each other by the adhesive.

Two joint parts 5a, 5b for joining the tapered portion 12 of the core shaft 1 and the tubular body 2 are formed inside the tubular body 2. The joint parts 5a, 5b are formed of a metal solder such as a silver solder, a gold solder, zinc, a Sn—Ag alloy, or an Au—Sn alloy, and the tapered portion 12 of the core shaft 1 and the tubular body 2 are fixed to each other by the metal solder. The joint parts 5a, 5b may be formed of an adhesive such as an epoxy adhesive, and the tapered portion 12 of the core shaft 1 and the tubular body 2 may be fixed to each other by the adhesive.

The inner tubular body 8 is shorter than the tubular body 2, and is wound around the outer side of the core shaft 1 so as to cover the outer periphery of from the small diameter portion 11 to a part of the tapered portion 12 of the core shaft 1. Accordingly, the tubular body 2 and the inner tubular body 8 overlap the outer side of the core shaft 1 only at the distal end portion of the guide wire 10.

A distal end of the inner tubular body 8 is fixed to the distal tip 3, and a proximal end of the inner tubular body 8 is fixed to the tapered portion 12 of the core shaft 1 by a joint part 5c. The joint part 5c may be formed of a metal solder such as a silver solder, a gold solder, zinc, a Sn—Ag alloy, or an Au—Sn alloy, or may be formed of an adhesive such as an epoxy adhesive.

The inner tubular body 8 may be a single coil formed into a hollow cylindrical shape by spirally winding one wire having a circular cross-section, or may be a hollow twisted wire coil formed into a hollow cylindrical shape by a twisted wire that is obtained by twisting a plurality of wires. The inner tubular body 8 may be configured by combining a single coil and a hollow twisted wire coil. The inner tubular body 8 can be formed of, for example, stainless alloys (SUS302, SUS304, SUS316, and the like), superelastic alloys such as a Ni—Ti alloy, a piano wire, a nickel-chromium alloy, radiotransparent alloys such as a cobalt alloy, gold, platinum, tungsten, and radiopaque alloys such as an alloy containing these elements (for example, a platinum-nickel alloy). The material of the inner tubular body 8 is not limited thereto, and may be formed of known materials other than the above-described materials. In the modification, the entire inner tubular body 8 is formed as a single member made of the same material, and the outer diameter thereof is configured to be constant from the distal end to the proximal end.

An outer peripheral surface of the tubular body 2 is partitioned into a first surface area 21 located on the distal end side and a second surface area 22 located closer to the proximal end side than the first surface area 21, and the surface property of the first surface area 21 is different from the surface property of the second surface area 22. The tubular body 2 having the first surface area 21 and the second surface area 22 on the outer peripheral surface is provided on the outer side of the core shaft 1. Accordingly, the guide wire 10 includes the first area 10a having a first surface property, and further includes the second area 10b having a second surface property and located closer to the proximal end side than the first area 10a. The first area 10a of the guide wire 10 is an area including the distal end of the guide wire 10.

The first surface property of the first area 10a is that the first area 10a has a first frictional resistance value R₁ when a frictional wear test is performed for the first area 10a. The second surface property of the second area 10b is that the second area 10b has a second frictional resistance value R₂ when the frictional wear test is performed for the second area 10b under the same conditions as those of the first area 10a. In the guide wire 10, the first frictional resistance value R₁ is larger than the second frictional resistance value R₂. A high friction area and a low friction area can be provided on the distal end side and the proximal end side of the guide wire 10, respectively, by controlling the surface property of the first area 10a and the surface property of the second area 10b. Accordingly, the advance of the knuckle can be controlled. That is, in the guide wire 10, the high friction area and the low friction area are provided on the distal end side (the first area 10a) and the proximal end side (the second area 10b) of the guide wire 10, respectively, so that the knuckle advance suppressing structure is realized.

In the guide wire 10, the first surface property of the first area 10a (the first surface area 21) may be that a surface friction coefficient of the first area 10a is a first friction coefficient μ₁. The second surface property of the second area 10b (the second surface area 22) may be that a surface friction coefficient of the second area 10b is a second friction coefficient μ₂. In this case, in the guide wire 10, the first friction coefficient μ₁ is larger than the second friction coefficient μ₂. As described above, the high friction area and the low friction area can be provided on the distal end side and the proximal end side of the guide wire 10, respectively, by controlling the surface property of the first area 10a and the surface property of the second area 10b.

A relationship between the above-described first friction coefficient μ₁ and the above-described second friction coefficient μ₂ may be adjusted, for example, by forming coating layers of different materials in the respective areas, or by forming a coating layer in only one of the areas. The above-described relationship may be adjusted by performing different surface processing treatments to the respective areas. The above-described relationship may be adjusted by forming coating layers of the same material in the respective areas and then performing a surface processing treatment on only the coating layer formed in one of the areas.

In the present embodiment, a first coating layer 6 is formed on the surface of the first area 10a of the guide wire 10, and a second coating layer 7 is formed on the second area 10b of the guide wire 10. Materials of the first coating layer 6 and the second coating layer 7 are selected such that the surface friction coefficient μ₁ of the first coating layer 6 is larger than the surface friction coefficient μ₂ of the second coating layer 7.

The first coating layer 6 is a silicone coating layer, and can be formed by a known coating forming method, for example, by applying a medical-grade silicone solution to the surface of the distal end side of the tubular body 2 and the distal tip 3. That is, in the present embodiment, the first surface property of the first area 10a is that the surface of the first area 10a is hydrophobic. In other words, it can be said that the silicone coating layer is formed as the first coating layer 6 on the surface of the first area 10a.

The second coating layer 7 is a hydrophilic coating layer. The hydrophilic coating layer can be formed by a known coating forming method. For example, a solution of a nonionic hydrophilic polymer such as polyvinyl alcohol, polyvinylpyrrolidone, polyethylene glycol, polyacrylamide, polymethylacrylamide, poly(2-hydroxyethyl methacrylate), or poly(N-hydroxyethylacrylamide), an anionic hydrophilic polymer such as polyacrylic acid, polymethacrylic acid, polymaleic acid, carboxymethyl cellulose, hyaluronic acid, or poly (2-acrylamido-2-methylpropanesulfonic acid), or a cationic hydrophilic polymer such as polyethyleneimine, polyallylamine, or polyvinylamine is applied to the surface of the proximal end side of the tubular body 2, the fixation portion 4, and the proximal end side of the large diameter portion 13 of the core shaft 1. That is, in the present embodiment, the second surface property of the second area 10b is that the surface of the second area 10b is hydrophilic. In other words, it can be said that the hydrophilic coating layer is formed as the second coating layer 7 on the surface of the second area 10b.

In the guide wire 10, the surface of the first area 10a located on the distal end side of the guide wire 10 is less slippery than the surface of the second area 10b located closer to the proximal end side than the first area 10a, so that the knuckle advance suppressing structure is realized. In such a guide wire 10, the first area 10a that is the high friction area is provided on the distal end side, and the second area 10b that is the low friction area is provided on the proximal end side. Accordingly, the guide wire 10 is easily bent up to the vicinity of a boundary between the first area 10a and the second area 10b, and the advance of the knuckle to the proximal end side from the vicinity of the boundary can be suppressed.

### <Modification of Structure of Guide Wire>

A modification of the structure of the guide wire used in the guide wire insertion method of the present disclosure will be described in connection with FIG. 2. In the above-described guide wire 10, the surface of the first area 10a located on the distal end side of the guide wire 10 is less slippery than the surface of the second area 10b located closer to the proximal end side than the first area 10a, so that the knuckle advance suppressing structure is realized. In a guide wire 10A of the modification described below, the rigidity of a first area 10Aa located on the distal end side of the guide wire and the rigidity of a second area 10Ab located closer to the proximal end side than the first area 10Aa are different from each other, so that the knuckle advance suppressing structure is realized.

FIG. 2 is illustrates a structure of the guide wire 10A according to the present embodiment. The guide wire 10A includes an elongated core shaft 1A and a tubular body 2A provided on the outer side of the core shaft 1A. A distal tip 3A for joining the core shaft 1A and the tubular body 2A is provided at the distal end of the guide wire 10A, and a fixation portion 4A for fixing the core shaft 1A and the tubular body 2A is provided at the proximal end of the tubular body 2A.

The core shaft 1A is an elongated member serving as a shaft of the guide wire 10A. As illustrated in FIG. 2, the core shaft 1A has a small diameter portion 11A on the distal end side and a large diameter portion 13A on the proximal end side, and has a tapered portion 12A between the small diameter portion 11A and the large diameter portion 13A. The outer diameter of the tapered portion 12A decreases from the proximal end side toward the distal end side. The core shaft 1A can be formed of, for example, materials such as stainless alloys (SUS302, SUS304, SUS316, and the like), superelastic alloys such as a Ni—Ti alloy, a piano wire, a nickel-chromium alloy, a cobalt alloy, and tungsten. The core shaft 1A is not limited thereto, and the core shaft 1A may be formed of other known materials as long as the core shaft 1A itself can be prevented from being cut and the distal end portion can be rotated.

The small diameter portion 11A, the tapered portion 12A, and the large diameter portion 13A of the core shaft 1A have rigidities different from each other due to the difference in diameters. The small diameter portion 11A has a columnar shape in which the outer diameter is constant from the distal end to the proximal end, and the moment of inertia of area is constant in the entire small diameter portion 11A. The large diameter portion 13A has a columnar shape in which the outer diameter is constant from the distal end to the proximal end, and the moment of inertia of area is constant in the entire large diameter portion 13A. The tapered portion 12A has a circular truncated cone shape in which the outer diameter is gradually enlarged from the distal end toward the proximal end, and the moment of inertia of area also gradually increases from the distal end toward the proximal end. Since the small diameter portion 11A of the core shaft 1A serves as the shaft of the guide wire 10A, the guide wire 10A is configured to vary the rigidity in the axial direction.

The tubular body 2A is wound around the core shaft 1A so as to cover the outer periphery of the small diameter portion 11A, the tapered portion 12A, and a part of the large diameter portion 13A of the core shaft 1A. The tubular body 2A may be a single coil formed into a hollow cylindrical shape by spirally winding one wire having a circular cross-section, or may be a hollow twisted wire coil formed into a hollow cylindrical shape by a twisted wire that is obtained by twisting a plurality of wires. The tubular body 2A may be configured by combining a single coil and a hollow twisted wire coil. The tubular body 2A can be formed of, for example, stainless alloys (SUS302, SUS304, SUS316, and the like), superelastic alloys such as a Ni—Ti alloy, a piano wire, a nickel-chromium alloy, radiotransparent alloys such as a cobalt alloy, gold, platinum, tungsten, and radiopaque alloys such as an alloy containing these elements (for example, a platinum-nickel alloy). The tubular body 2A is not limited thereto, and may be formed of known materials other than the above-described materials. In the present embodiment, the entire tubular body 2A is formed as a single member made of the same material, and the outer diameter thereof is configured to be constant from the distal end to the proximal end.

The distal tip 3A for joining the core shaft 1A and the tubular body 2A is formed at the distal end of the guide wire 10A (that is, the distal end of the core shaft 1A). The distal tip 3A is formed of a metal solder such as a silver solder, a gold solder, zinc, a Sn—Ag alloy, or an Au—Sn alloy, and the distal end of the core shaft 1A and the distal end of the tubular body 2A are fixed to each other by the metal solder. The distal tip 3A may be formed of an adhesive such as an epoxy adhesive, and the distal end of the core shaft 1A and the distal end of the tubular body 2A may be fixed to each other by the adhesive.

The fixation portion 4A for fixing the core shaft 1A and the tubular body 2A is formed at the proximal end of the tubular body 2A. The fixation portion 4A is formed of a metal solder such as a silver solder, a gold solder, zinc, a Sn—Ag alloy, or an Au—Sn alloy, and the proximal end of the tubular body 2A is fixed to the large diameter portion 13A of the core shaft 1A by the metal solder. The fixation portion 4A may be formed of an adhesive such as an epoxy adhesive, and the large diameter portion 13A of the core shaft 1A and the proximal end of the tubular body 2A may be fixed to each other by the adhesive.

Two joint parts 5Aa, 5Ab for joining the tapered portion 12A of the core shaft 1A and the tubular body 2A are formed inside the tubular body 2A. The joint parts 5Aa, 5Ab are formed of a metal solder such as a silver solder, a gold solder, zinc, a Sn—Ag alloy, or an Au—Sn alloy, and the tapered portion 12A of the core shaft 1A and the tubular body 2A are fixed to each other by the metal solder. The joint parts 5Aa, 5Ab may be formed of an adhesive such as an epoxy adhesive, and the tapered portion 12A of the core shaft 1A and the tubular body 2A may be fixed to each other by the adhesive.

A coating layer such as a silicone coating layer or a hydrophilic coating layer may be formed on the surface of the tubular body 2A, the distal tip 3A, the fixation portion 4A, and the surface of the proximal end side of the large diameter portion 13A of the core shaft 1A.

In the guide wire 10A, the rigidity of the first area 10Aa located on the distal end side of the guide wire 10A and the rigidity of the second area 10Ab located closer to the proximal end side than the first area 10Aa are different from each other, so that the knuckle advance suppressing structure is realized. The first area 10Aa includes the small diameter portion 11A of the core shaft 1A, and the second area 10Ab includes the tapered portion 12A and the large diameter portion 13A of the core shaft 1A. In such a guide wire 10A, the first area 10Aa having a low rigidity is provided on the distal end side, and the second area 10Ab having a high rigidity is provided on the proximal end side. Accordingly, the guide wire 10A is easily bent up to the vicinity of a boundary B between the first area 10Aa and the second area 10Ab, and the advance of the knuckle to the proximal end side from the vicinity of the boundary can be suppressed.

### <Guide Wire Insertion Method>

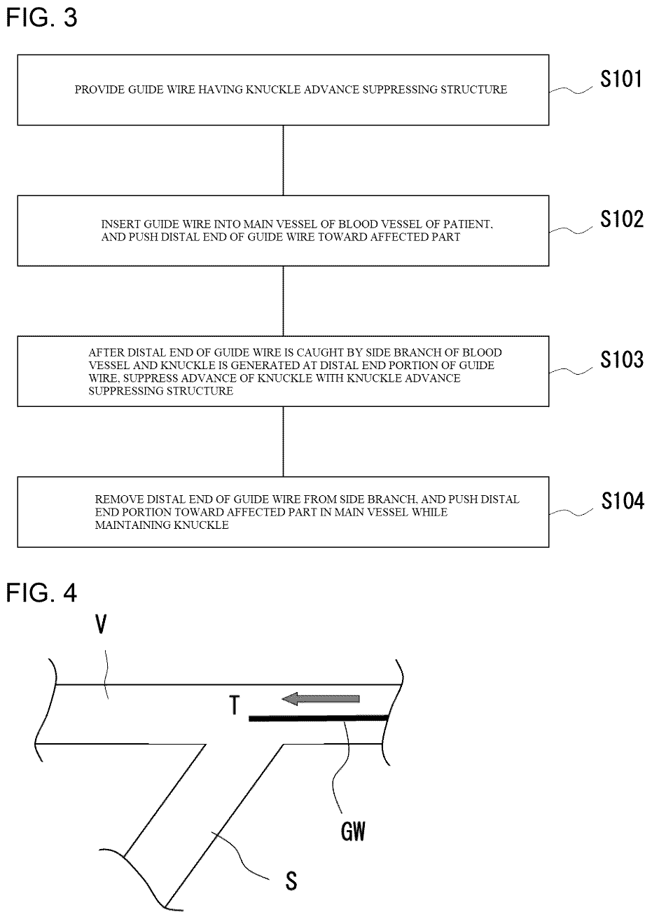

Next, a guide wire insertion method of the present disclosure will be described with reference to the drawings. The guide wire insertion method is a method that can be performed in treatment of ischemic cardiac disease in a cardiac coronary artery in which a guide wire is inserted for guiding a catheter or the like for percutaneous coronary intervention (PCI) to a constricted part in a coronary artery into a blood vessel having a main vessel and a side branch branched from the main vessel. As illustrated in FIG. 3, specifically, the guide wire insertion method includes the following steps.

In step S101, a guide wire GW having a knuckle advance suppressing structure is prepared. As the guide wire GW having a knuckle advance suppressing structure, the above-described guide wire 10 (the surface of the first area 10a located on the distal end side of the guide wire 10 is less slippery than the surface of the second area 10b located closer to the proximal end side than the first area 10a, so that the knuckle advance suppressing structure is realized) and the above-described guide wire 10a (the rigidity of the first area 10Aa located on the distal end side of the guide wire and the rigidity of the second area 10Ab located closer to the proximal end side than the first area 10Aa are different from each other, so that the knuckle advance suppressing structure is realized) can be used.

In step S102, a professional inserts the guide wire GW into a main vessel V of a blood vessel of a patient, and pushes a distal end T of the guide wire GW toward an affected part. As illustrated in FIG. 4, the distal end T of the guide wire GW does not form a knuckle at this stage, and the guide wire GW proceeds through the main vessel V of the blood vessel while maintaining the substantially linear shape.

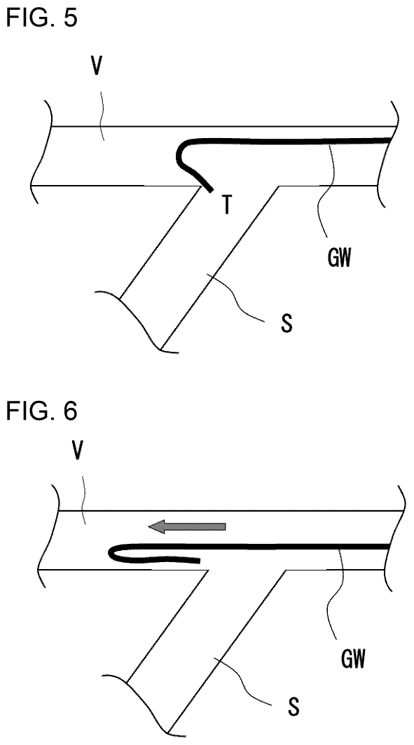

In step S103, after the distal end T of the guide wire GW is caught by a side branch S of the blood vessel and a knuckle is generated at the distal end portion of the guide wire GW, the advance of the knuckle is suppressed with the knuckle advance suppressing structure. FIG. 5 illustrates a state in which the knuckle is generated at the distal end portion of the guide wire GW. The professional operates the guide wire GW to cause the distal end T of the guide wire GW to be caught by the side branch S of the blood vessel, and pushes the guide wire GW in this state to generate the knuckle at the distal end portion of the guide wire GW.

In step S104, the professional removes the distal end T of the guide wire GW from the side branch S, and pushes the distal end portion toward the affected part in the main vessel V while maintaining the knuckle. FIG. 6 illustrates a state in which the distal end portion of the guide wire GW is pushed forward while maintaining the state in which the knuckle is generated. The professional operates the guide wire GW to remove the distal end T of the guide wire GW from the side branch S of the blood vessel, and pushes the guide wire GW in this state to deliver the distal end portion of the guide wire GW to the affected part in the main vessel V. Since the guide wire GW has the knuckle advance suppressing structure, the knuckle does not excessively advance even when the professional pushes the guide wire GW.

In Step S104, the professional may continue to push the distal end portion of the guide wire GW forward in the main vessel V to a site in which a blood vessel inner diameter is 4 to 6 mm. A general blood vessel diameter of the main vessel V is 4 to 6 mm. Therefore, in order for the professional to deliver the distal end portion of the guide wire GW to the affected part, it is necessary to continue to push the distal end portion of the guide wire GW forward in the main vessel V to the site in which the blood vessel inner diameter is 4 to 6 mm.

In Step S104, the professional may continue to push the distal end portion of the guide wire GW forward in the main vessel V to a site in which the blood vessel inner diameter is 1 to 2 mm. In order for the professional to deliver the distal end portion of the guide wire GW to the periphery of the main vessel V, to the professional can continue to insert the distal end portion of the guide wire GW to the site in which the blood vessel inner diameter is 1 to 2 mm.

According to such a guide wire insertion method, the professional causes the distal end T of the guide wire GW to be caught by the side branch S of the blood vessel to generate the knuckle, and causes the distal end portion of the guide wire GW to proceed toward the affected part in this state. Accordingly, the distal end T of the guide wire GW does not migrate into a microscopic blood vessel, and the distal end T is not pressed against a blood vessel wall in the process of pushing the guide wire GW forward. Therefore, a trouble such as dissociation or perforation of the blood vessel wall is less likely to occur. When the professional pushes the guide wire GW forward in the state in which the knuckle is generated at the distal end portion of the guide wire GW, the generation of the knuckle may advance to the proximal end side of the guide wire GW (professional's hand side) due to the resistance received from the blood vessel wall. Since the guide wire GW has the knuckle advance suppressing structure, further advance of the knuckle is suppressed, so that the load on the blood vessel wall is reduced. When the guide wire GW in the state in which the knuckle is generated at the distal end portion is pushed forward, the contact between the guide wire GW and the blood vessel wall is not a point contact but a line contact. Therefore, the load on the blood vessel wall is also suppressed due to a decrease in surface pressure.

The guide wire insertion method according to the present disclosure and the structure of the guide wire used in the method have been described above with reference to the drawings. The present disclosure is not limited to the above-described embodiments, and various modifications can be made.

What is claimed is:

1. A guide wire insertion method for inserting a guide wire into a blood vessel of a patient that includes a main vessel and a side branch branched from the main vessel, the method comprising:
providing a guide wire having a knuckle advance suppressing structure;
inserting the guide wire into the main vessel and pushing a distal end of the guide wire toward an affected part in the main vessel;
after the distal end of the guide wire is caught by the side branch, generating a knuckle at a distal end portion of the guide wire, and
removing the distal end of the guide wire from the side branch, and pushing the distal end portion toward the affected part in the main vessel while maintaining the knuckle,
wherein the knuckle advance suppressing structure is configured to suppress advance of the knuckle as the distal end portion is pushed toward the affected part in the main vessel.

2. The guide wire insertion method according to claim 1, wherein the knuckle advance suppressing structure includes a surface of a first area located on a distal end side of the guide wire that is less slippery than a surface of a second area located closer to a proximal end side than the first area.

3. The guide wire insertion method according to claim 1, wherein the knuckle advance suppressing structure includes a first area, located on a distal end side of the guide wire, with a first rigidity and a second area, located closer to a proximal end side than the first area, with a second rigidity, and wherein the first rigidity and the second rigidity are different from each other.

4. The guide wire insertion method according to claim 1, wherein the method is performed in treating ischemic cardiac disease in a cardiac coronary artery.

5. The guide wire insertion method according to claim 1, wherein the distal end portion is pushed forward in the main vessel to a site in which the main vessel has an inner diameter in a range of from 4 to 6 mm.

6. The guide wire insertion method according to claim 1, wherein the distal end portion is pushed forward in the main vessel to a site in which the main vessel has an inner diameter in a range of from 1 to 2 mm.

7. The guide wire insertion method according to claim 1, wherein the guide wire is a workhorse guide wire.

8. The guide wire insertion method according to claim 3, wherein the first rigidity is lower than the second rigidity.

9. The guide wire insertion method according to claim 1, wherein the guide wire includes an elongated core shaft and a tubular body that is joined to the core shaft at a distal end side of the guide wire, and wherein the knuckle advance suppressing structure is part of the tubular body.
